(19) United States
(12) Patent Application Publication
(10) Pub. No.: US 2023/0138203 A1
(43) Pub. Date: May 4, 2023

(54) ELECTRIC AND FLUIDIC CABLES

(71) Applicant: ABIOMED, Inc., Danvers, MA (US)

(72) Inventors: Christopher Theodore Bazdanes, Danvers, MA (US); Robert Schaefer, Danvers, MA (US); Vincent Roy, Danvers, MA (US); Emilia Jahangir, Danvers, MA (US)

(73) Assignee: ABIOMED, Inc., Danvers, MA (US)

(21) Appl. No.: 17/978,619

(22) Filed: Nov. 1, 2022

Related U.S. Application Data
(60) Provisional application No. 63/274,829, filed on Nov. 2, 2021.
(60) Provisional application No. 63/275,278, filed on Nov. 3, 2021.

(51) Int. Cl. A61M 60/13; A61M 60/216; A61M 60/829; A61M 60/508; A61M 60/411; A61M 60/855

(52) U.S. Cl. CPC A61M 60/13; A61M 60/216; A61M 60/829; A61M 60/508; A61M 60/411; A61M 60/855

(57) ABSTRACT

A cable that includes a first tubular body defining a first lumen therethrough, a tubular purge body disposed within the first lumen and extending along at least a portion of a length of the first lumen, the tubular purge body defining a purge lumen arranged to transfer a fluid therethrough, and at least one tubular electrical wire body disposed within the first lumen, each tubular electrical wire body defining an electrical wire lumen therethrough for receiving one or more electrical wires. The cable may also include one or more overmolds and one or more cases. The cable may be operably connected between a handle for a medical device and a controller.

### CROSS-REFERENCE TO RELATED APPLICATIONS

The present application claims priority to U.S. Provisional Patent Application Nos. 63/274,829, filed Nov. 2, 2021, and 63/275,278, filed Nov. 3, 2021, both of which are hereby incorporated by reference in their entirety.

### TECHNICAL FIELD

The present disclosure relates to cables configured to allow both electrical wiring and fluids to safely and securely be passed through the cable, such as to an intravascular blood pump used to support flow in a patient's blood vessel.

### BACKGROUND

Present day medical treatments, such as cardiovascular treatments, may involve the use of numerous medical devices, fluid lines, and the like. These fluid lines may provide, e.g., necessary fluid to the patient, may be removing or recirculating fluids from the patient (such as blood), or may be providing fluid needed by a medical device, such as a purge fluid. For example, in some blood pumps, a purge fluid may be deployed to keep blood from entering the pump mechanism and to mitigate the effects of blood and/or bio-deposit buildup on the pump mechanisms.

### BRIEF SUMMARY

According to a first aspect of the present disclosure, a cable may be arranged to operably connect a blood pump to a controller, where the cable may include a first tubular body defining a first lumen therethrough, a tubular purge body disposed within the first lumen and extending along at least a portion of a length of the first lumen, the tubular purge body defining a purge lumen arranged to transfer a fluid therethrough, and at least one tubular electrical wire body disposed within the first lumen, each tubular electrical wire body defining an electrical wire lumen therethrough for receiving one or more electrical wires.

In some embodiments, the first tubular body may comprise a plurality of layers, such as an inner binder layer and an outer jacket. In some embodiments, the inner binder layer may comprise or consist of polytetrafluoroethylene (PTFE), the outer jacket may comprise or consist of a thermoplastic polyurethane, or a combination thereof. Filler may be present within the first lumen and may be disposed around at least a portion of the tubular purge body and the tubular electrical wire body. In some embodiments, the filler comprises or consists of polytetrafluoroethylene (PTFE).

In some embodiments, the tubular purge body may comprise a plurality of layers, such as an inner binder layer and an outer jacket. In some embodiments, the inner binder layer may comprise or consist of polytetrafluoroethylene (PTFE), the outer jacket may comprise or consist of a thermoplastic polyurethane, or a combination thereof.

In some embodiments, a proximal portion of the tubular purge body may be operably connected to an input connector, where the input connector may be configured to provide a sterile attachment to a fluid source. In some embodiments, the fluid source may be removably attachable from the input connector. In some embodiments, the proximal portion of the tubular purge body may be connected to a pressure storage set (PSS) assembly which may comprise or be operably connected to the input connector.

In some embodiments, the tubular purge body may be configured to extend through the first lumen from the distal end of the first tubular body to the proximal end of the first tubular body. In some embodiments, openings in the first tubular body may be created to allow the tubular purge body to pass through a sidewall of the first tubular body at an intermediate location. In some embodiments, the first tubular body may comprise an outer surface defining a first opening extending through a sidewall to an inner surface of the first tubular body, the inner surface defining the first lumen, the tubular purge body configured to enter the first lumen through the first opening.

Various configurations of internal cable organization may be utilized. In some embodiments, a plurality of tubular electrical wire bodies may be present, including a first tubular electrical wire body and a second tubular electrical wire body. In some embodiments, the first tubular electrical wire body may be configured to receive at least one electrical wire in electrical communication with a motor. In some embodiments, a plurality of electrical wires may extend through the electrical wire lumen of one or more of the at least one tubular electrical wire bodies. In some embodiments, a plurality of electrical wires may extend through each electrical wire lumen. In some embodiments, at least one electrical wire extending through a second tubular electrical wire body may be in electrical communication with a sensor. In some embodiments, all electrical wires are within a single tubular electrical wire body. In some embodiments, each tubular electrical wire body may contain the same number of electrical wires. In some embodiments, a number of electrical wires in the first tubular electrical wire body may be different from a number of electrical wires in the second tubular electrical wire body. In some embodiments, the first tubular electrical wire body contains 2-4 electrical wires, and the second tubular electrical wire body contains 3-5 electrical wires. In some embodiments, one or more of the electrical wires may comprise copper. In some embodiments, each electrical wires may comprise copper. In some embodiments, each electrical wire may comprise or consist of copper surrounded by an electrically insulating sheath. In some embodiments, filler may be disposed around at least a portion of at least one electrical wire.

In some embodiments, the cable may also contain a tubular optical fiber body within the first lumen, where the tubular optical fiber body defines an optical fiber lumen therethrough. In some embodiments, the tubular optical fiber body may comprise or consist of polytetrafluoroethylene (PTFE).

In some embodiments, the cable may extend from a controller to a handle (e.g., a handle for controlling an insertable or implantable medical device). In some embodiments, the controller is configured to control a fluid flowing through a purge lumen extending through the cable, receive optical data through an optical fiber slidably positioned within the optical fiber lumen, and control a motor through at least one electrical wire.

In some embodiments, the cable may also comprise a first overmold, which is connected to a portion of the first tubular body at a location distal to an opening defined by an outer surface of the first tubular body extending to the first lumen and connected to a portion of both the first tubular body and the first tubular body at a location proximal to the defined opening. In some embodiments, the cable may also comprise a second overmold, the second overmold connected to a portion of the first tubular at a location proximal from the first overmold.

The cable may also comprise a case configured to surround at least a portion of the first tubular body proximal to the first overmold, the case may be configured to connect to the first overmold such that any component surrounded by the case cannot be twisted, pulled apart, or pushed apart.

In some embodiments, the case may be a two-part case, which may comprise or consist of a polycarbonate. In some embodiments, a first part (e.g., first body) of the two-part case may be configured to connect to a second part (e.g., second body) of the two-part case by inserting one or more pins on the first part into a corresponding boss on the second part, and by one or more raised portions on an internal surface of the first part interface with a plurality of depressions on an external surface of the second part. In some embodiments, the case may be configured to define a first port surrounding a first overmold, a second port for the first tubular body distal from a second overmold and proximal from the first overmold, and a third port for allowing a fluid source to operably connect to the tubular purge body through an input connector.

According to a second aspect of the present disclosure, a system may comprise a controller, a pump, and a cable as described previously, where the cable operably connects the pump to the controller. In some embodiments, the cable may extend from a handle to the controller.

According to a third aspect of the present disclosure, an overmold may be provided for a first tubular body defining a first lumen therethrough, the first tubular body having a second tubular body within the first lumen, the second tubular body defining a second lumen therethrough, the second tubular body configured to enter the first lumen through an opening defined by an outer surface of the first tubular body and extending to the first lumen. The overmold may comprise a distal portion connected to a portion of the first tubular body at a location distal to the defined opening, and a proximal portion connected to a portion of both the first tubular body and the second tubular body at a location proximal to the defined opening, where the proximal portion is configured to connect to a case such that the case cannot twist, push, or pull apart from the overmold.

In some embodiments, the case may be a two-part case comprising a first part and a second part, where the first part is in contact with a first portion of the overmold, and the second part is in contact with a second portion of the overmold.

In some embodiments, the first tubular body may comprise a plurality of layers, such as an inner binder layer and an outer jacket. In some embodiments, the inner binder layer may comprise or consist of polytetrafluoroethylene (PTFE), the outer jacket may comprise or consist of a thermoplastic polyurethane, or a combination thereof. Filler may be present within the first lumen and may be disposed around at least a portion of the second tubular body and at least one additional tubular body extending through at least a portion of the first lumen. In some embodiments, the filler comprises or consists of polytetrafluoroethylene (PTFE).

In some embodiments, the second tubular body may comprise a plurality of layers, such as an inner binder layer and an outer jacket. In some embodiments, the inner binder layer may comprise or consist of polytetrafluoroethylene (PTFE), the outer jacket may comprise or consist of a thermoplastic polyurethane, or a combination thereof.

In some embodiments, a proximal portion of the second tubular body may be operably connected to an input connector, where the input connector may be configured to provide a sterile attachment to a fluid source. In some embodiments, the fluid source may be removably attachable from the input connector. In some embodiments, the proximal portion of the tubular purge body may be connected to a physiological saline solution (PSS) assembly which may comprise or be operably connected to the input connector.

In some embodiments, at least one additional tubular body may extend through the first lumen from a proximal end to a distal end, each additional tubular body defining a lumen extending therethrough. In some embodiments, the at least one additional tubular body may comprise a plurality of additional tubular bodies, including a first additional tubular body defining a first additional lumen and a second additional tubular body defining a second additional lumen. In some embodiments, at least one electrical wire may extend through one or more of the at least additional tubular bodies.

In some embodiments, one or more electrical wires may be in electrical communication with a motor. In some embodiments, at least one electrical wire may extend through the second additional tubular body, and one of those electrical wires may be in electrical communication with a sensor.

In some embodiments, the first and second additional tubular bodies may be configured to receive the same number of electrical wires. In some embodiments, a number of electrical wires in the first additional tubular body may be different from a number of electrical wires in the second additional tubular body. In some embodiments, the number of electrical wires in the first additional tubular body may be 2-4, and the number of electrical wires in the second additional tubular body may be 3-5. In some embodiments, a plurality of electrical wires may extend through one of the at least one additional tubular body. In some embodiments, a plurality of electrical wires may extend through each additional tubular body. In some embodiments, one or more of the electrical wires may comprise copper. In some embodiments, each electrical wires may comprise copper. In some embodiments, each electrical wire may comprise or consist of copper surrounded by an electrically insulating sheath. In some embodiments, filler may be disposed around at least a portion of at least one electrical wire.

In some embodiments, the at least one additional tubular body may comprise a tubular optical fiber body defining an optical fiber lumen configured to slidably receive an optical fiber therethrough. In some embodiments, the optical fiber lumen is configured to slidably receive a plurality of optical fibers therethrough.

In some embodiments, the first tubular body may extend proximally from a handle. In some embodiments, the first tubular body may extend from a handle to a controller, where the overmold may be positioned between the handle and the controller. In some embodiments, the controller may be configured to control a fluid flowing through the purge lumen, receive optical data through an optical fiber slidably positioned within the optical fiber lumen, and control a motor through at least one electrical wire.

According to a fourth aspect of the present disclosure, a case, such as a two-part case, may be configured to contain a PSS assembly and clamp around an overmold, where the overmold surrounds a portion of a first tubular body and a second tubular body, the second tubular body being connected to the PSS assembly. In some embodiments, the case may be arranged to chemically and/or mechanically protect the PSS. For example, in some embodiments, the case may facilitate user interaction with the PSS.

In some embodiments, the case may include two bodies that are arranged to engage with one another. For example, the first body of the two-part case may include a pin, a boss, or a combination thereof, and at least one raised portion on an interior surface of the first part. In such an example, the second body of the two-part case may include a pin, a boss, or a combination thereof, and a plurality of depressed portions on an exterior surface of the second body to engage with the first body. Each pin or boss is configured to align with a boss or pin, respectively, on the opposite part, and the at least one raised portion is configured to interact with each depressed portion such that the two parts cannot be pulled apart.

### BRIEF DESCRIPTION OF DRAWINGS

FIG. 1 is a depiction of an embodiment of a prior art system.

FIG. 2A is a depiction of an embodiment of a disclosed cable within a system.

FIG. 2B is a depiction of an additional embodiment of a disclosed cable within a system.

FIGS. 3A-3E are cross-sectional views of various embodiments of a disclosed cable.

FIG. 4A is a cross-sectional side view of an embodiment of a first overmold.

FIG. 4B is a cross-sectional side view of an embodiment of a first and second overmold.

FIG. 5A is a depiction of an embodiment of a cable, overmold, and case.

FIG. 5B is a depiction of an embodiment of a first part of a two-part case.

FIG. 5C is a depiction of an embodiment of a second part of a two-part case.

FIG. 5D is a cross-sectional view of an embodiment of a snap fit connection between a first part and a second part of a two-part case.

FIG. 6 is an exploded view of an embodiment of a cable, overmold, and case.

FIG. 7 is an exploded view of an embodiment of a cable and a case.

FIG. 8 is an exploded view of an embodiment of a cable, an overmold, and a case.

FIG. 9 is an exploded view of an embodiment of a cable and a case.

FIG. 10 is an exploded view of an embodiment of a cable, and a case.

FIGS. 11A and 11B are exploded views of an embodiment of a cable and a case.

FIG. 12 is a perspective view of a case according to one embodiment.

FIGS. 13A and 13B are a depiction of an embodiment of a case.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Blood pumps of different types are known, such as axial blood pumps, centrifugal blood pumps, or mixed-type blood pumps, where the blood flow is caused by both axial and radial forces. One such example of a blood pump is the Impella® line of blood pumps (e.g., Impella 2.5®, Impella CP®, Impella 5.5®, etc.), which are products of Abiomed Inc. of Danvers, Mass. Intravascular blood pumps may be inserted into a patient's vessel, such as via the aorta, by means of a catheter.

Such blood pumps may involve the use of numerous medical devices, fluid lines, and the like. These fluid lines may be providing, e.g., necessary fluid to the patient, may be removing or recirculating fluids from the patient (such as blood), or may be providing fluid needed by a medical device, such as a purge fluid. For example, in some blood pumps, a purge fluid may be deployed to keep blood from entering the pump mechanism and to mitigate the effects of blood and/or bio-deposit buildup on the pump mechanisms.

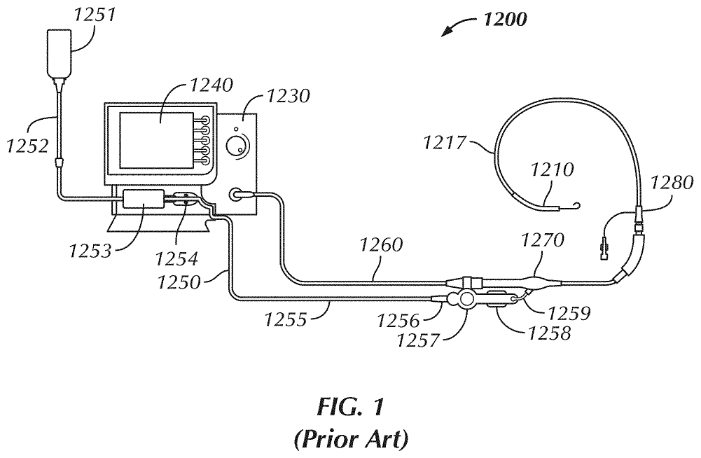

In existing systems, see, e.g., FIG. 1, a blood pump assembly 1200 may include a blood pump 1210 fluidically connected to a purging device 1250. The blood pump assembly 1200 also may include a controller 1230 (e.g., an Automated Impella Controller® from Abiomed, Inc., Danvers, Mass.), a display 1240, a connector cable 1260, a plug 1270, and a repositioning unit 1280. As shown, the controller 1230 may include a display 1240. Controller 1230 may monitor and controls blood pump 1210. During operation, purging device 1250 may deliver a purge fluid to blood pump 1210 through catheter tube 1217, such as to prevent blood from entering the motor (not shown) within motor housing 1216. In some implementations, the purge fluid comprises a dextrose solution (e.g., 5% dextrose in water with 25 or 50 IU/mL of heparin). Connector cable 1260 may provide an electrical connection between blood pump 1210 and controller 1230. Plug 1270 connects catheter tube 1217, purging device 1250, and connector cable 1260. In some embodiments, plug 1270 may include a memory for storing operating parameters in case the patient needs to be transferred to another controller. Repositioning unit 1280 may be used to reposition blood pump 1210. As shown in this view, the fluid line may be separate from the connector cable having one or more electrical wires.

The inventors have recognized that fluid lines in blood pumps and/or other medical devices may need to remain thin, light, and flexible to be useful during the procedures, but this results in situations where a patient's movements and/or movements of a user such as a medical provider may cause mechanical loads that lead to breakage, separation, or disconnection of some fluid lines, or result in kinks being formed in the fluid lines. A means for overcoming these hurdles is therefore useful and desirable. For example, as described herein, a cable may be configured to fluidically and electrically couple a blood pump and/or another medical device to a controller (or other suitable device). In some embodiments, the cable also may include one or more optical wires to optically connect the blood pump to the controller. As also described herein, the medical device assembly may include a case arranged to protect the PSS and cable. In some embodiments, an overmold may extend over a portion of the cable.

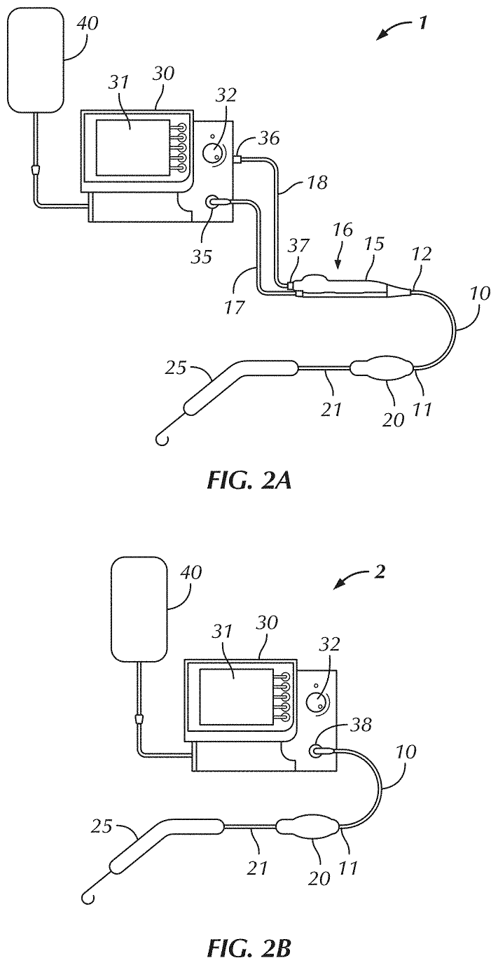

Referring to FIG. 2A, a system 1 according to one embodiment is depicted, with the system 1 having a controller 30, a medical device, such as a blood pump 25, and a cable 10 that operably connects the blood pump to the controller. In such embodiment, as described herein, the cable may be configured to receive fluid and electrical wire(s) through the cable. In some embodiments, the cable also may be configured to receive optical fiber(s) through the cable. The medical device may be, e.g., a catheter-based blood pump. As will be appreciated, the current disclosure is not limited to any particular blood pump or other suitable medical device. In some embodiments, the blood pump 25 may include a catheter 21 and a handle 20. The fluid, electrical signals, and optionally optical signals may pass from the cable 10 through the handle 20, through catheter 21, and to the blood pump 25. For example, in some embodiments, the fluid may include a purge fluid, and the handle may include a coupling between a purge lumen in the catheter 21 and a purge lumen in the cable 10. Similarly, in some embodiments, one or more electrical and/or optical connections may be formed within the handle 20 to allow electrical and/or optical signals to pass from the cable 10, through the handle 20, and through the catheter 21 to the blood pump 25. In other embodiments, one or more fluid lumens, electrical wires, and/or optical fibers may extend continuously from the cable 10, through the handle 20 and catheter 21, to the blood pump 25. Thus, depending on the particular embodiment, the handle 20 may, or may not, include one or more fluidic, electrical, and/or optical connections.

In some embodiments, the cable may be configured to have a distal end 11 connected to a handle 20 and the proximal end 12 operably connected to an input connector 37, the input connector configured to provide a sterile attachment to a fluid source. In some embodiments, the fluid source may be removably attached to the input connector. In some embodiments, a proximal end (or proximal portion) of the tubular purge body may be connected to a pressure storage set (PSS) assembly 16 that may comprise the input connector 37.

As described herein, the cable 10 may be configured to allow a fluid source 40, which may be removably attached, to provide fluids to the patient or the blood pump 25 through the cable 10. In some embodiments, the cable 10 may branch into two separate lines 17, 18. The cable 10 may operably connect the pump 25 to the controller 30 through, e.g., one or more connectors 35, 36. In some embodiments, the connector 36 may interface with the pressure line (not shown) of a purge cassette. In some embodiments, the system may have an input connector 37 that allows a second line 18 to be removably connected, where the second line may be configured to provide a fluid to the cable. In some embodiments, the first line 17 may electrically and/or optically connect the cable to the controller 30.

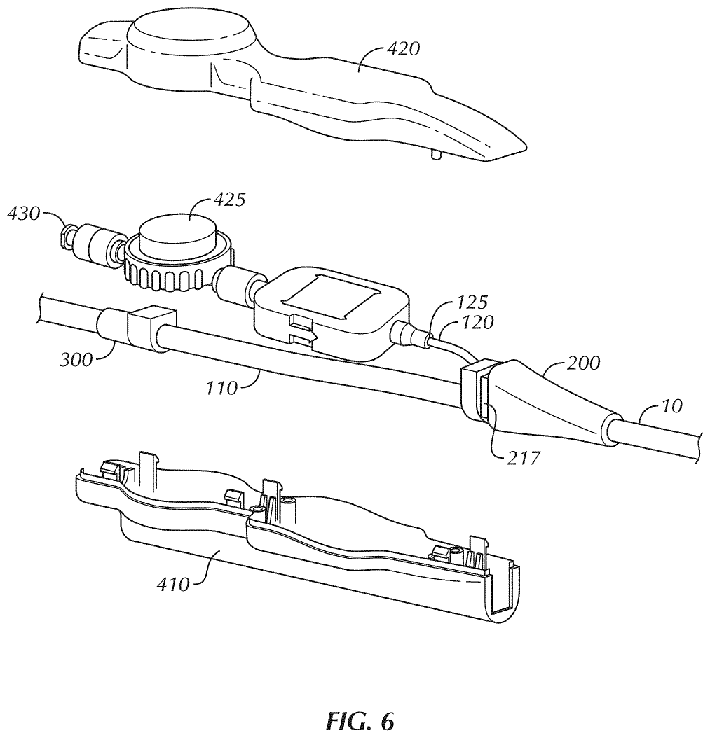

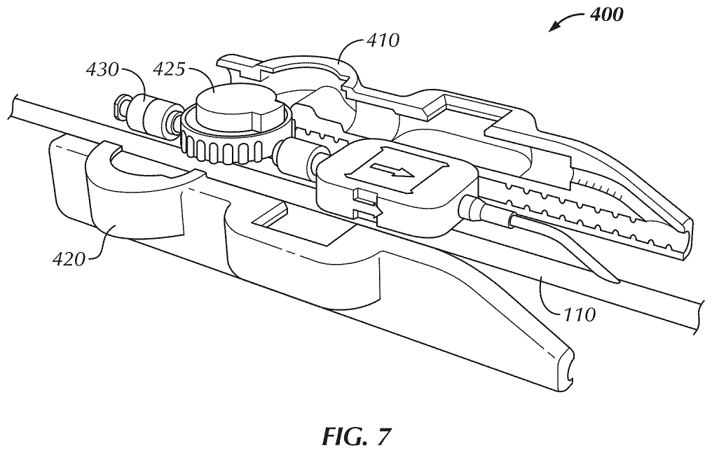

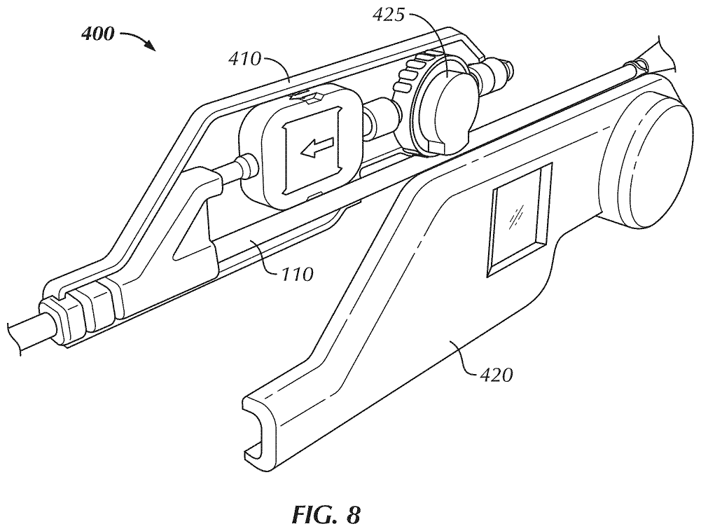

In some embodiments, the handle may include a case 15 (see, e.g., FIG. 2A) that encompasses at least a portion of the cable 10. As will be appreciated, the cable may branch into the separate lines which are protected via the case. For example, as shown in FIGS. 6 and 7, branches may be formed independent of the case in which they are housed.

Referring to FIG. 2B, an alternate system 2 is depicted, where the cable 10 extends from the handle 20 to the controller 30 and connects to the controller via a connector 38. In some embodiments, the cable may be configured to have the distal end 11 connected to a handle 20 and the proximal end 12 connected or operably connected to an input connector 38, the input connector configured to provide a sterile attachment to a fluid source. In some embodiments, the fluid source may be removably attached to the input connector.

Cable

To enable the cable 10 to contain electrical wiring and allow fluids to pass through the cable simultaneously, cable 10 may advantageously be configured in certain arrangements.

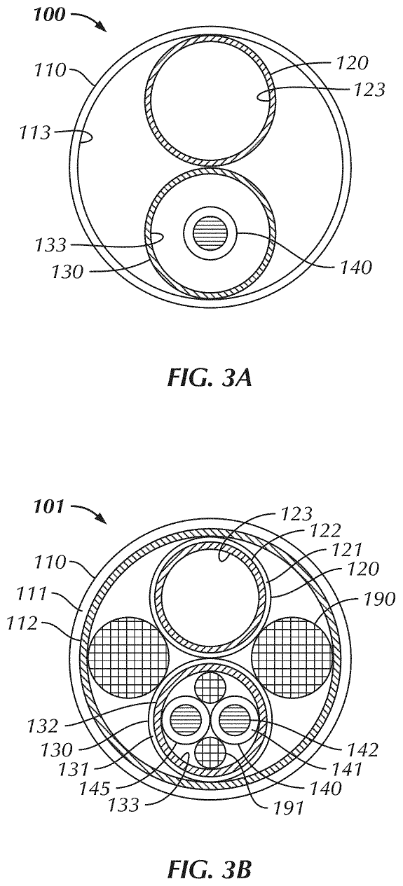

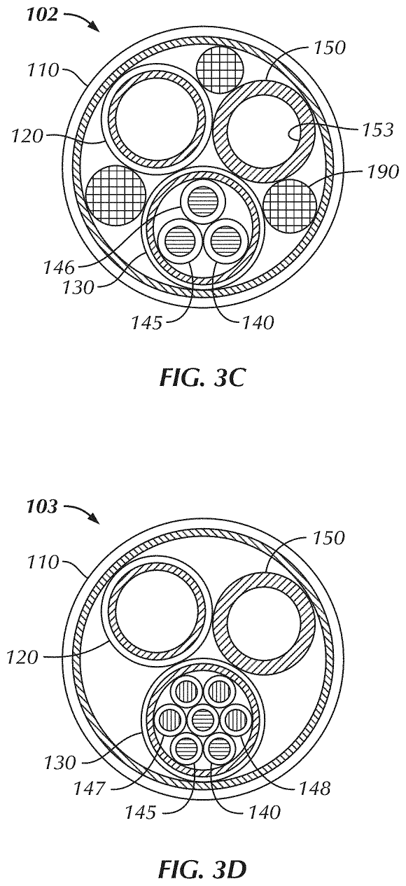

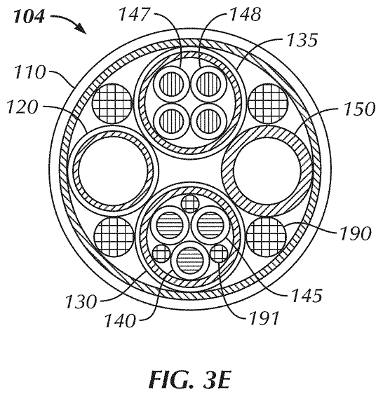

Referring to FIG. 3A, a cross section of an embodiment of a cable 100 can be seen. Cable 100 may comprise a first, outer, tubular body 110 having a distal end and a proximal end, the first tubular body having an inner surface 113 defining a first lumen therethrough. Cable 100 may also have a fluid line, such as a tubular purge body 120 disposed within the first lumen and extending along at least a portion of a length of the first lumen, the tubular purge body 120 having an inner surface 123 defining a purge lumen arranged to transfer a fluid therethrough. Cable 100 may also have at least one tubular electrical wire body 130 disposed within the first lumen, each tubular electrical wire body having an inner surface 133 defining an electrical wire lumen therethrough for receiving one or more electrical wires 140.

As will be appreciated, although the tubular purge body and the tubular electrical wire body is shown as having the same outer diameter, the diameter of the tubular bodies may vary in other embodiments. As will be further appreciated, the tubular purge body and tubular electrical body may have any suitable arrangement in the first tubular body. In some embodiments, the tubular purge body and the tubular electrical body may be attached to one another and/or to the first tubular body, although the tubular purge body and the tubular electrical body may move freely relative to one another in the first tubular body.

In some embodiments, the first tubular body may have a nominal outer diameter between 3 mm and 8 mm, such as between 5.5 mm-6.5 mm.

Referring to FIG. 3B, a cross section of an embodiment of a cable 101 can be seen. In some embodiments, the first tubular body 110 may comprise a plurality of layers 111, 112. In some embodiments, this plurality of layers may comprise an inner binder layer 112 and an outer jacket 111. In some embodiments, the inner binder layer 112 of the first tubular body may comprise or consist of polytetrafluoroethylene (PTFE) and the outer jacket 111 of the first tubular body may comprise or consist of a thermoplastic polyurethane.

In some embodiments, a cable may also comprise filler 190 within the first lumen. The filler may comprise or consist of polytetrafluoroethylene (PTFE). In some embodiments, the filler 190 may be disposed around at least a portion of the tubular purge body 120 and the tubular electrical wire body 130. In such embodiments, the filler may be configured to maintain a position of the tubular purge body relative to the tubular electrical wire body.

In some embodiments, the tubular purge body 120 also may comprise a plurality of layers 121, 122. In some embodiments, this plurality of layers may comprise an inner binder layer 122 and an outer jacket 121. In some embodiments, the inner binder layer 122 of the tubular purge body may comprise or consist of polytetrafluoroethylene (PTFE) and the outer jacket 121 of the tubular purge body may comprise or consist of a thermoplastic polyurethane.

In some embodiments, the tubular purge body may be configured to extend through the first lumen from the distal end of the first tubular body to the proximal end of the first tubular body.

In some embodiments, the at least one tubular electrical wire body 130 may be configured to receive a plurality of electrical wires 140, 145. In some embodiments, filler 191 may be disposed around at least a portion of at least one electrical wire 140, 145. In some embodiments, each electrical wire may comprise copper, although the wire may be formed of other suitable materials. In some embodiments, each electrical wire comprises a conductive core 142 (which may comprise copper) surrounded by an insulating jacket 141.

Referring to FIGS. 3C and 3D, a cross section of embodiments of a cable 102, 103 can be seen. In some embodiments, at least one of the electrical wires 140, 145, 146 within the tubular electrical wire body 130 may be in electrical communication with a motor (e.g., the motor of an axial or centrifugal blood pump). In some embodiments, each of the electrical wires within the tubular electrical wire body 130 may be in electrical communication with a motor. In some embodiments some of the electrical wires 140, 145 within the tubular electrical wire body 130 may be in electrical communication with a motor, while one or more other electrical wires 147, 148 within the tubular electrical wire body are in electrical communication with a different component or device, such as one or more sensors.

In some embodiments, the cable may also comprise a tubular optical fiber body 150 within the first lumen, the tubular optical fiber body having an inner surface 153 defining an optical fiber lumen therethrough, the optical fiber lumen configured to slidably receive an optical fiber. In some embodiments, the tubular optical fiber body may comprise or consist of polytetrafluoroethylene (PTFE). In some embodiments, the optical fiber lumen has an inner diameter of between 0.8 mm and 1.5 mm. In some embodiments, the tubular optical fiber body may have a wall thickness between 0.1 and 0.4 mm. As will be appreciated, the outer diameter of the tubular optical body may be the same as the tubular purge body and/or the tubular electrical body, although one or more of the tubular bodies may have a different diameter than the other.

Referring to FIG. 3E, a cross section of an embodiment of a cable 104 can be seen. In some embodiments, the cable may comprise a plurality of tubular electrical wire bodies 130, 135, including a first tubular electrical wire body 130 and a second tubular electrical wire body 135. In some embodiments, at least one electrical wire body 147, 148 in the second tubular electrical wire body are in electrical communication with a device or component (such as a sensor) other than the motor of a blood pump. In some embodiments, a plurality of electrical wires may extend through each electrical wire lumen. In some embodiments, a number of electrical wires 145 in the first tubular electrical wire body 130 may be the same as the number of electrical wires 147, 148 in the second tubular electrical wire body 135. In some embodiments, a number of electrical wires 145 in the first tubular electrical wire body 130 may be different from a number of electrical wires 147, 148 in the second tubular electrical wire body 135. In some embodiments, the number of electrical wires in the first tubular electrical wire body 130 is 2-4, and number of electrical wires in the second tubular electrical wire body 135 is 3-5.

As will be appreciated, in some embodiments, each of the tubular purge body, the tubular electrical wire body, and the tubular optical fiber body may extend an entire length of the outer first tubular body (e.g., between the tubular and proximal ends). In other embodiments, one or more of the tubular bodies may extend only partially through the outer first tubular body. For example, in some embodiments, the tubular purge body may exit the first tubular body at a position between the distal and proximal ends.

Controller

Referring to FIGS. 2A and 2B, in some embodiments, the controller 30 may be configured to control fluid flowing through the purge lumen, receive optical data through an optical fiber slidably positioned with in the optical fiber lumen and control a motor through at least one electrical wire. In such embodiments, the controller may include at least one processor and at least one non-transitory computer readable medium containing instructions that, when executed, causes the at least one processor to control a pump, valve, or combination thereof to cause a fluid to flow through the purge lumen, receive optical data from an optical fiber, and control a motor (such as the rotational speed of a motor).

In some embodiments, the at least one processor is configured to cause adjustments to current flowing to a motor.

In some embodiments, the controller also controls a display 31 (such as a touchscreen display), and or may be configured to receive user input from one or more buttons, knobs, or other controls 32 for, e.g., selecting modes of operation.

Overmold

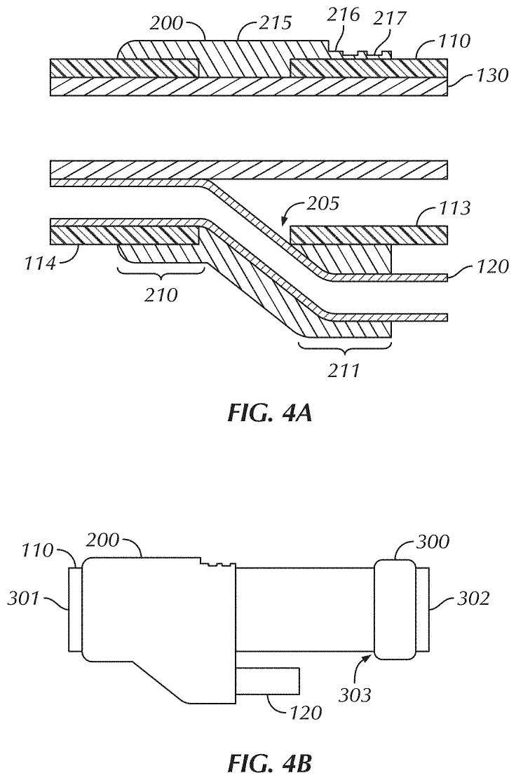

Referring to FIG. 4A, in some embodiments, the cable may comprise a first overmold 200. The first overmold 200 may be connected to a portion 210 of the first tubular body 110 at a location distal from a defined opening 205 extending from an outer surface 114 of the first tubular body to an inner surface 113 of the first tubular body. The first overmold also may be connected to a portion 211 of both the tubular purge body 120 and the first tubular body 110 at a location proximal to the defined opening.

In some embodiments, the first overmold 200 may be configured to connect to a case. In some embodiments, at least one surface 216 may be recessed from an outer surface 215 of the first overmold. The depth of the recession may be configured to allow an outer surface of a case to be adjacent to and substantially coplanar with the outer surface 215 of the first overmold. In some embodiments, the first overmold may also have additional cutouts or depressions 217 that are configured to allow a case to be firmly connected to the first overmold (e.g., via snap fittings, pressure fit, slidably connected, etc.). The case may then have respective features on an internal surface that allow it to interact with the cutouts or depressions 217. In this manner, a case may be connected to the first overmold such that the case cannot twist apart, push apart, or pull apart from the first overmold.

Referring to FIG. 4B, in some embodiments, the cable may have a distal end 301 and a proximal end 302 and may comprise the first overmold 200 and a second overmold 300. The second overmold 300 may be positioned proximal to the first overmold 200, and an inner surface of the overmold 303 may be connected to the first tubular body 110.

In some embodiments, the overmold may assist in creating branches in the fluid line. For example, in some embodiments, the overmold may allow one of the tubular bodies to exit the cable at a location between the proximal end and the distal end. For example, in some embodiments, the first tubular body may exit the first tubular body at or via the overmold.

Case

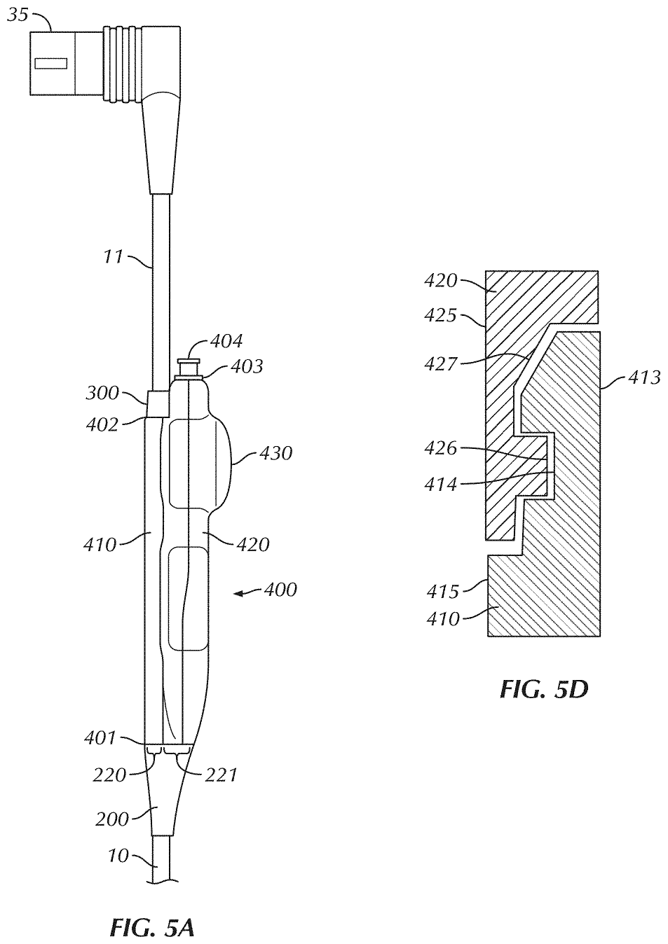

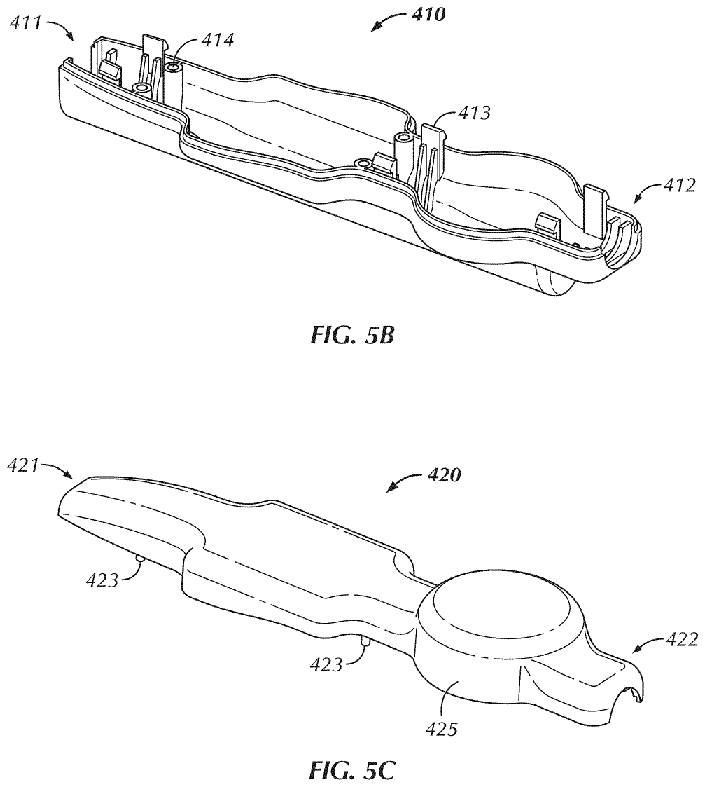

Referring to FIG. 5A, an embodiment of a cable operably connected to a connector 35, the cable comprising a case 400 and overmolds 200, 300, can be seen. As depicted, in some embodiments, a case 400 may be configured to surround at least a portion of the first tubular body 10, 11 proximal to the first overmold 200, the case configured to connect to the first overmold 200 such that any component surrounded by the case cannot be twisted, pulled apart, or pushed apart.

In some embodiments, the case may be a two-part case having first and second bodies 410, 420. In some embodiments, each body may be formed of a polycarbonate material. In some embodiments, the case 400 is configured to contain a PSS assembly 430. The two parts of the case may be configured to clamp together around an overmold 200, the overmold surrounding a portion of a first tubular body and a second tubular body (such as a tubular purge body), where the second tubular body may be connected to the PSS assembly 430.

In some embodiments, the case 400 defines a first port 401 surrounding a first overmold 200, a second port 402 for the first tubular body 11 distal from a second overmold 300 and proximal from the first overmold 200, and a third port 403 for allowing a fluid source to operably connect to the tubular purge body through an input connector 404.

In some embodiments, the first body 410 of the case may be in contact with a first portion 220 of the first overmold 200, and the second body 420 is in contact with a second portion 221 of the overmold.

As will be appreciated, the cable need not include an overmolded portion. Accordingly, in such embodiments, the first port 401 may surround only the cable 110 (see, e.g., FIG. 7). As also shown in FIG. 7, the port may be connected to a first channel 440 for receiving the cable. The case also may include a second channel 442 for receiving a branch of the cable (e.g., a fluid line connected to a purge cassette). As will be appreciated the channels may have any suitable arrangement (e.g., straight and/or curved). In some embodiments, the first and second bodies of the case (as described herein) may cooperate to form the channel within which the cable and/or branches extend. The channels also may be formed in only one of the bodies. As will be further appreciated, in other embodiments, the channels may be housed in the case in other suitable manners.

Referring to FIGS. 5B-5D, the first body 410 of the case may be configured to connect to the second body 420 using a plurality of pins 423 and bosses 414, and a plurality of snap-fit connections 413. In some embodiments, a first body 410 may comprise a pin, a boss, or a combination thereof, and a plurality of depressed portions on an exterior surface of the second part, and a second body 420 may include either a pin, a boss, or a combination thereof, and at least one raised portion on an interior surface of the first part, where each pin or boss is configured to align with a boss or pin, respectively, on the opposite body, and the raised portion is configured to interact with each depressed portion such that the two parts cannot be pulled apart.

In some embodiments, the case can be connected by inserting one or more pins 423 on the second body into a corresponding boss 414 on the first body, and by one or more raised portions 426 on an internal surface 427 of the second part 425 interfacing with a plurality of depressions 414 on an external surface 415 of the first part.

As will be appreciated, other suitable fasteners may be used to couple the first and second bodies of the case together. The first and second bodies also may be connected via other suitable arrangements in other.

Referring to FIG. 6, it can be seen that in some embodiments, the two bodies 410, 420 of the case are configured to contain a PSS assembly 425 and clamp around an overmold 200.

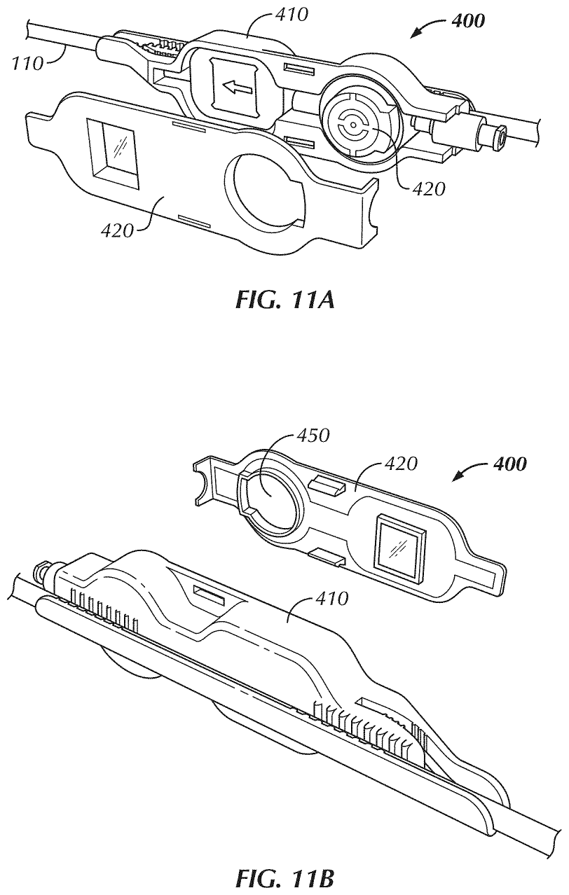

As shown in FIGS. 7-11B, the case may have other suitable arrangements for housing the cable and PSS assembly. As shown in these examples (e.g., FIG. 7), the first and second bodies may have similar constructions (e.g., mirror images of each other) and cooperate to form the housing for receiving the PSS assembly and cable. In some embodiments (see, e.g., FIGS. 11A and 11B, one body may include a cover that is attached to the second body.

In some embodiments, the case may include one or more windows 450 (see, e.g., FIG. 11B) for viewing the PSS assembly, a portion of the cable, and/or the overmold. In some embodiments, the windows may be formed in a single body (see, e.g., FIGS. 11A and 11B), while in other embodiments, the first and second bodies may cooperate to form one or more windows in the case.

In some embodiments, the case may include one or more doors through which a clinician may access cables, cassettes, or other components housed within the cable.

As shown in FIGS. 7-11B, the PSS assembly and cable may have any suitable arrangement in the case. For example, in some embodiments, the PSS assembly may be positioned on top of the cable (see, e.g., FIGS. 6 and 7). In such embodiments, as seen in FIG. 7, the X-Y plane of the PSS assembly does not intersect with a central axis of the first tubular body. That is, a bottom surface of the PSS assembly may be directed towards the first tubular body. In some embodiments, the cases may be configured to have two parts that each is configured to conform with either a first or second portion (e.g., a left or right portion) of the PSS assembly.

In other embodiments (see, e.g., FIG. 8), the cable and the PSS assembly may be placed side by side. As seen in this view, in some embodiments, the X-Y plane of the PSS assembly may intersect with a central axis of the first tubular body. That is, a left or right surface of the PSS assembly may be directed towards the first tubular body, and the bottom surface may be orthogonal to the central axis. In some embodiments, the cases may be configured to have two parts that each conform with either a bottom portion or a top portion of the PSS assembly.

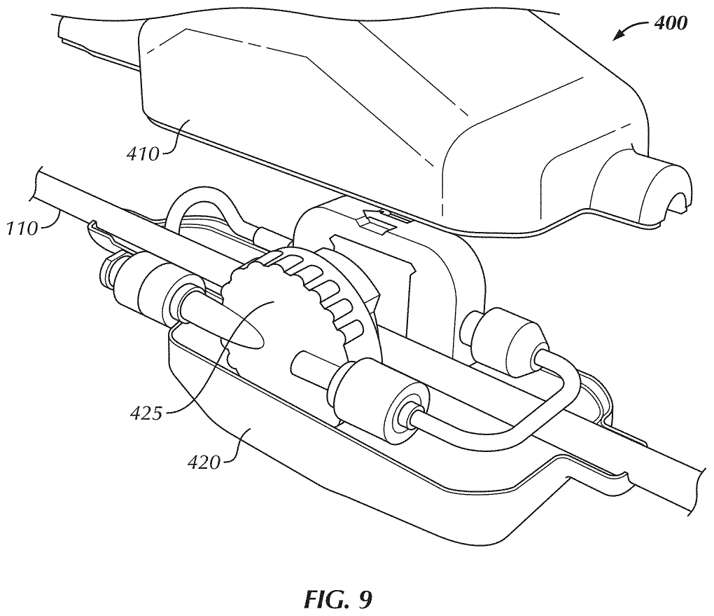

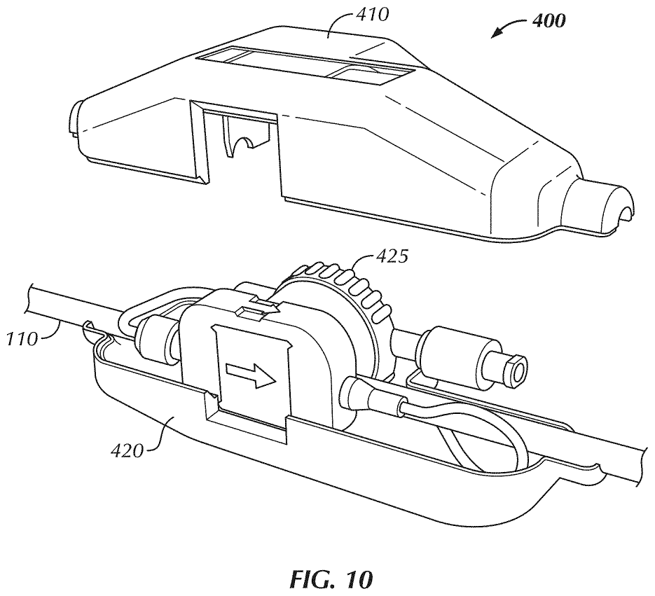

In other embodiments (see, e.g., FIGS. 9 and 10), the PSS assembly may be folded around the cable. For example, as seen in these views, in some embodiments, the PSS assembly is not arranged in a linear fashion. In some embodiments, a first portion of the PSS assembly may be present on one side of the first tubular body, and a second portion of the PSS assembly may be present on a second side of the first tubular body.

The case also may be configured such that a majority of the PSS assembly is partially encompassed by a first part of the case, and a second part of the case is substantially acting as a lid or cover when the two parts are combined (see FIGS. 11A and 11B).

In some embodiments, the case may include a first region to include a cassette attachable to the fluid line.

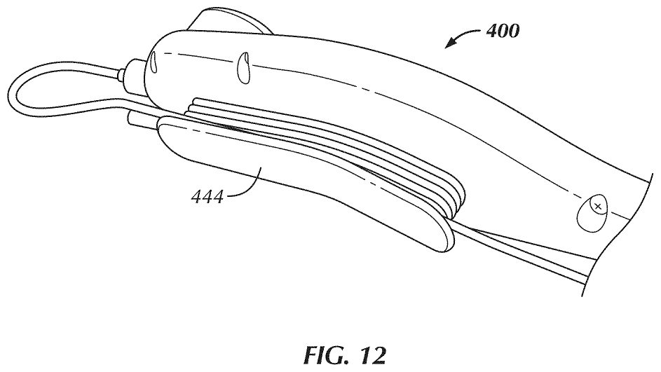

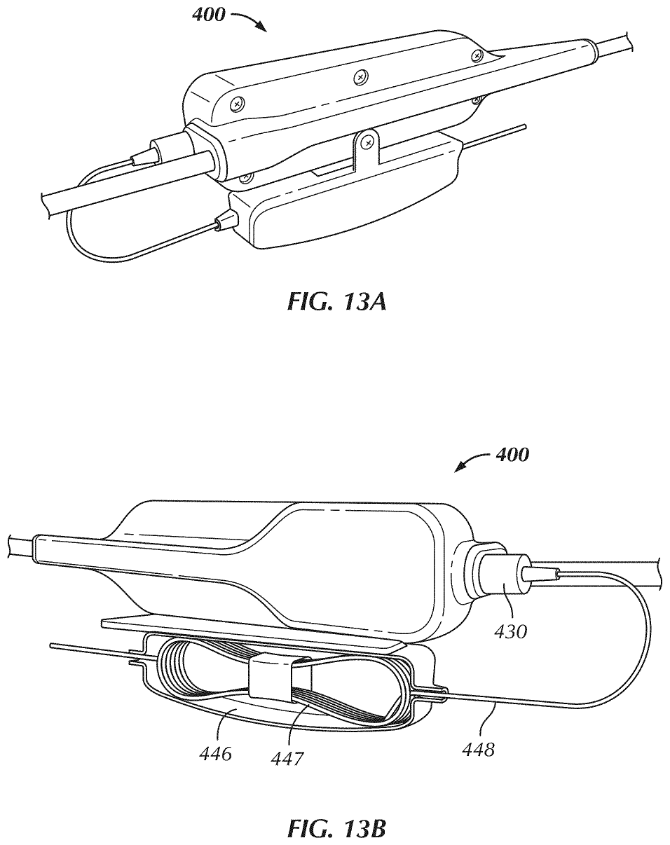

In some embodiments, the case may be arranged to retain a portion of cable extending from the case. In some embodiments, the cable may be removable attachable to the case for extending between the case and controller. For example, as shown in FIG. 12, the case may include a holder 444 around which a length of cable may be wrapped. As will be appreciated, the holder may be removably attached to one of the bodies, may be fixedly attached thereto, or maybe integrally formed therewith. As shown in FIGS. 13A and 13B, the case also may include a compartment 446 for holding a length of cable. For example, a spool of cable 447 may be held in the compartment in some embodiments. In some embodiments, the cable 448 that forms the spool of cable 447 may extend from an input connector 430. In some embodiments, the cable that forms the spool of cable does not extend from an input connector.

The case also may have other stiffening and/or reinforcement features (e.g., crush ribs). In some embodiments, the cable and/or PSS may be positioned at or near the ribs. In some embodiments, the crush ribs may be positioned around a portion of the cable and/or PSS when the case is assembled.

The overmold, as described herein, may surround a portion of a first tubular body 110 and a second tubular body (such as tubular purge body 120). The first part 410 may be configured to connect with a depression or groove 217 (see, e.g., FIG. 6) in the overmold, to prevent the case from twisting, pushing, or pulling apart. A proximal end 125 of the second tubular body (here, tubular purge body 120) may be connected to a PSS assembly, which may comprise an input connector 430. The input connector may be removably connectable to a fluid source.

A distal end of the cable 10 may be connected to a handle (not shown). The handle may comprise, e.g., circuitry protected by an outer housing. A proximal end of the cable may be directly or indirectly connected to one or more connectors 35 and through the connector 35 to a controller. The connector may be configured to provide electrical and optical connections to the controller.

Although the case is shown and described as being a two-part case, in other embodiments, the case may include three of more bodies that corporate to form a compartment for holding the PSS and the cable, and in some embodiments, an overmold.

Those skilled in the art will recognize or be able to ascertain using no more than routine experimentation many equivalents to the specific embodiments of the invention described herein. Such equivalents are intended to be encompassed by the following claims.

1. A cable arranged to operably connect a blood pump to a controller, the cable comprising:
a first tubular body having a distal end and a proximal end, the first tubular body defining a first lumen therethrough,
a tubular purge body disposed within the first lumen and extending along at least a portion of a length of the first lumen, the tubular purge body defining a purge lumen arranged to transfer a fluid therethrough; and
at least one tubular electrical wire body disposed within the first lumen, each tubular electrical wire body defining an electrical wire lumen therethrough for receiving one or more electrical wires.

2. The cable according to claim 1, wherein the first tubular body comprises a plurality of layers.

3. (canceled)

4. (canceled)

5. The cable according to claim 1, further comprising filler within the first lumen.

6. The cable according to claim 5, wherein the filler is disposed around at least a portion of the tubular purge body and the tubular electrical wire body.

7. (canceled)

8. The cable according to claim 1, wherein the tubular purge body comprises a plurality of layers.

9. (canceled)

10. (canceled)

11. The cable according to claim 1, wherein a proximal portion of the tubular purge body is operably connected to an input connector, the input connector configured to provide a sterile attachment to a fluid source.

12. (canceled)

13. The cable according to claim 11, wherein the proximal portion of the tubular purge body is connected to a pressure storage set (PSS) assembly comprising the input connector.

14. The cable according to claim 1, wherein the first tubular body comprises an outer surface defining a first opening extending to an inner surface of the first tubular body, the inner surface defining the first lumen, the tubular purge body configured to enter the first lumen through the first opening.

15. The cable according to claim 1, wherein the tubular purge body is configured to extend through the first lumen from the distal end of the first tubular body to the proximal end of the first tubular body.

16. The cable according to claim 1, wherein the at least one tubular electrical wire body is configured to receive at least one electrical wire in electrical communication with a motor.

17. The cable according to claim 1, wherein the at least one tubular electrical wire body comprises a plurality of tubular electrical wire bodies, including a first tubular electrical wire body and a second tubular electrical wire body.

18. The cable according to claim 17, wherein at least one electrical wire extending through the second tubular electrical wire body is in electrical communication with a sensor.

19. (canceled)

20. (canceled)

21. (canceled)

22. (canceled)

23. (canceled)

24. (canceled)

25. The cable according to claim 1, further comprising a tubular optical fiber body within the first lumen, the tubular optical fiber body defining an optical fiber lumen therethrough.

26. (canceled)

27. The cable according to claim 1, wherein the cable extends from a handle to a controller.

28. The cable according to claim 27, wherein the controller is configured to control a fluid flowing through the purge lumen, receive optical data through an optical fiber slidably positioned within the optical fiber lumen, and control a motor through at least one electrical wire.

29. The cable according to claim 1, further comprising a first overmold, the first overmold connected to a portion of the first tubular body at a location distal to a defined opening extending from an outer surface of first tubular body to an inner surface of the first tubular body, and connected to a portion of both the tubular purge body and the first tubular body at a location proximal to the defined opening.

30. The cable according to claim 29, further comprising a second overmold, the second overmold connected to a portion of the first tubular body at a location proximal from the first overmold.

31. The cable according to claim 29, further comprising a case configured to surround at least a portion of the first tubular body proximal to the first overmold, the case configured to connect to the first overmold such that any component surrounded by the case cannot be twisted, pulled apart, or pushed apart.

32-35. (canceled)

36. A system comprising:
a controller;
a pump; and
a cable according to claim 1, the cable operably connecting the pump to the controller.

37. The system according to claim 36, wherein the cable extends from a handle to the controller.

38. An overmold for a first tubular body defining a first lumen therethrough, the first tubular body having a second tubular body within the first lumen, the second tubular body defining a second lumen therethrough, the second tubular body configured to enter the first lumen through an opening defined by an outer surface of the first tubular body, the opening extending to the first lumen, the overmold comprising:
a distal portion connected to a portion of the first tubular body at a location distal to the defined opening; and
a proximal portion connected to a portion of both the first tubular body and the second tubular body at a location proximal to the defined opening, the proximal portion configured to connect to a case such that the case cannot twist, push, or pull apart from the overmold.

39-63. (canceled)

64. A two-part case configured to contain a PSS assembly and clamp around an overmold, the overmold surrounding a portion of a first tubular body and a second tubular body, the second tubular body being connected to the PSS assembly, the two-part case comprising:
a first part comprising either a pin, a boss, or a combination thereof, and a plurality of depressed portions on an exterior surface of the first part; and
a second part comprising a pin, a boss, or a combination thereof, and at least one raised portion on an interior surface of the second part;
wherein each pin or boss is configured to align with a boss or pin, respectively, on an opposite part, and wherein the at least one raised portion is configured to interact with each depressed portion such that the first part and the second part cannot be pulled apart.
